(19) United States
(12) Patent Application Publication
(10) Pub. No.: US 2022/0355289 A1
(43) Pub. Date: Nov. 10, 2022

(54) SYSTEMS AND METHODS FOR MULTI-TARGET DEPOSITION AND ASSAYS

(71) Applicant: THE REGENTS OF THE UNIVERSITY OF MICHIGAN, Ann Arbor, MI (US)

(72) Inventors: Max Shtein, Ann Arbor, MI (US); Y. Eugene Shteyn, Cupertino, CA (US); Steven Morris, Ann Arbor, MI (US); Brian lezzi, Ann Arbor, MI (US); Siddharth Borsadia, Ann Arbor, MI (US); Joanna Ciatti, Ann Arbor, MI (US); Katie Wei, Ann Arbor, MI (US)

(21) Appl. No.: 17/604,919

(22) PCT Filed: Apr. 20, 2020
(86) PCT No.: PCT/US20/28993

§ 371 Date: Oct. 19, 2021

Related U.S. Application Data
(60) Provisional application No. 62/836,403, filed on Apr. 19, 2019.

(51) Int. Cl. B01L 3/02; C12Q 1/02; B01L 3/00; B05D 1/00

(52) U.S. Cl. CPC B01L 3/0289; C12Q 1/025; B01L 3/5085; B01L 3/52; B05D 1/60; B01L 2200/16; B01L 2200/148; B01L 2200/141

(57) ABSTRACT

Disclosed herein is are methods and apparatuses for synthesizing deposited films of compounds (e.g., organic compounds such as a pharmaceutical active ingredient or a new chemical entity) on or in a variety of substrates, where such deposited compounds the desired stability under storage conditions, ease of handling, and yet enhanced dissolution properties when used in various assays. The disclosure further relates to methods of coating substrates, such as medical or diagnostic devices, with deposited films of organic compounds, as well as film-coated substrates.

### CROSS-REFERENCE TO RELATED APPLICATIONS

This application claims the benefit of U.S. Provisional Application Ser. No. 62/836,403, filed Apr. 19, 2019, the entirety of which is hereby incorporated herein by reference.

### STATEMENT OF GOVERNMENT SUPPORT

This invention was made with government support under EEC1647837, EFMA1240264, 1727894, 1827123, awarded by the National Science Foundation. The government has certain rights in the invention.

### BACKGROUND

### Technical Field

The present disclosure relates to methods and apparatuses for synthesizing deposited films of compounds (e.g., organic compounds such as a pharmaceutical active ingredient or a new chemical entity), where such deposited compounds exhibit controlled dissolution behavior (e.g. faster or slower dissolution, controlled amount). The disclosure further relates to methods of coating substrates, such as medical or diagnostic devices, with deposited films of organic compounds, as well as film-coated substrates.

### Description of Related Technology

Modern drug research and development uses high throughput screening (HTS) to test active ingredients (including drugs). It is common to use micro-well plates into which active ingredients, cells, and/or other ingredients are dispensed using manual or automated pipetting apparatus, or more recently, using acoustic focusing droplet ejection technology. The injected ingredients are typically taken from stock solutions and diluted to desired concentration. Oftentimes, there are problems with adequately controlling concentration. These occur with the stock solutions themselves, as well as with controlling concentration of the ingredients in the wells. For example, the article “Overcoming Problems of Compound Storage in DMSO: Solvent and Process Alternatives” by T. J. WAYBRIGHT, J. R. BRITT, and T. G. McCLOUD notes that in 26% of the −300,000 compound library at the National Cancer Institute (NCI), the concentration of the supernatant in a stock solution was not the expected value after prolonged storage (e.g. months or several years). The precipitation or degradation of solute (e.g. drug or other ingredient), or the varying concentration due to hygroscopicity of DMSO (a typical solvent) causes poor reproducibility and other problems in the HTS assays they are used in.

It is therefore desirable to have a way to improve the reliability of assays and generate the desired concentration of ingredient, maximize the concentration of ingredient, and/or broaden the flexibility of applications of the HTS paradigm to performing biological, pharmacological, and other assays.

An alternative to solution-based storage of drugs is dry storage. The benefits of dry storage are believed to include a decreased rate of decomposition, no precipitation, and decreased cross-contamination due to spillage (i.e., “creep”) between wells. For dry storage of test plates to be a useable and successful technique in an HTS environment, it must be possible for compounds with a wide variety of physical properties to be rapidly brought into solution. However, many compounds are not highly soluble even in pure DMSO or other commonly-used solvents, and dissolution can be worsened when the compound to be solubilized has been vacuum-dried. Moreover, a complication to dry storage is that the compound may not adhere in the well and may move around during handling, becoming lost or cross-contaminating adjacent wells. For this reason, it is desirable to provide a platform, such as a printed film, that allows a compound to be stably stored over a period of time and readily solubilized before use.

### SUMMARY

In one aspect, described herein is a method of depositing a compound on a substrate comprising: (a) forming a compound vapor; (b) passing the compound vapor through a nozzle oriented towards the substrate; (c) depositing the compound on the substrate; (d) displacing one of the nozzle or the substrate relative to the other; and (e) repeating steps (c) and (d) as required to achieve a desired deposition protocol.

In another aspect, described herein is a testing environment having a printed surface, wherein the printed surface is prepared by: (a) forming a compound vapor; (b) passing the compound vapor through a nozzle oriented towards the surface; (c) depositing the compound on the substrate as a solid; (d) displacing one of the nozzle or the substrate relative to the other; and (e) repeating steps (c) and (d) as required to achieve a desired deposition protocol.

In another aspect, described herein is a method of screening an analyte comprising contacting the analyte with a testing environment described herein.

In another aspect, described herein is a testing station comprising: a compound reservoir configured to hold a compound vapor; a nozzle array coupled to the compound reservoir, the nozzle array comprising a plurality of nozzles configured to receive the compound vapor; a substrate positioned to receive the compound vapor from the nozzle array, the substrate having one or more deposition regions configured for deposition of the compound on the substrate; and a positioner for displacing at least one of the nozzle array or the substrate relative to the other for depositing the compound on the substrate according to a designed deposition protocol.

In another aspect, described herein are methods for high-throughput screening and high-throughput testing, and systems for carrying out the methods. In some aspects, a high-throughput screening method described herein comprises a preparation stage, a distribution stage, an application stage, an observation stage, and an analysis stage.

Further aspects and advantages will be apparent to those of ordinary skill in the art from a review of the following detailed description, taken in conjunction with the drawings. The description hereafter includes specific embodiments with the understanding that the disclosure is illustrative, and is not intended to be limited to the specific embodiments described herein.

### BRIEF DESCRIPTION OF THE DRAWINGS

This patent or application file contains at least one drawing executed in color. Copies of this patent or patent application publication with color drawing(s) will be provided by the United States Patent and Trademark Office upon request and payment of the necessary fee.

The figures described below depict various aspects of the system and methods disclosed herein. It should be understood that each figure depicts an embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

FIG. 1 is a schematic of an example vapor deposition device, in accordance with an example. A compound vapor (drug vapor) is jetted into a reservoir (left) with drugs and/or with solvents, wherein the compound vapor dissolves (right). A valve allows controlled introduction of the dissolved compound into a liquid handler, which can allow the compound to be introduced into a testing environment, characterized, stored, or further used in manufacturing processes.

FIG. 2 is a schematic of the use of the vapor deposition device of FIG. 1 to produce stocks of compounds in solution at a controlled concentration. Compound vapor (drug vapor) is jetted into a reservoir (left) with drugs and/or with solvents, wherein the compound vapor dissolves (right). This produces a high concentration “local stock solution” which can be redistributed by a liquid handler into well plates and further diluted to a desired concentration.

FIG. 3 is a schematic of the use of the vapor deposition device of FIG. 1 to produce deposit films of compound on a substrate: (A) compound vapor (e.g., a drug in vapor form) is jetted into a compartment to produce a solid film. (B) each compartment can contain more than one solid film of a different compound. (C) compound vapor can also be deposited on a post to produce a solid film on the post. (D) compound vapor can also be deposited on crystals or as part of a crystal growing process.

FIG. 4 is a schematic of the use of the vapor deposition device of FIG. 1 to produce (A) discrete patterns of a compound (e.g., a drug) on a substrate or (B) a continuous gradient of compound(s) across the substrate.

FIG. 5 is a schematic of the use of printed films in high throughput screening applications such as assays, in accordance with an example: (A) a compound vapor is jetted onto a substrate to produce a solid film of e.g., a drug. (Ba)-(Da): If the substrate is e.g., a well plate, a solvent is added to produce a solution of the compound in the solvent, a second component (e.g., cells) is added to the well and allowed to incubate or develop, and an observable result is detected using appropriate methods (e.g., spectroscopy). (Bb)-(db): If the substrate is e.g., a post, the post is introduced into an appropriate receiving vessel containing an analyte solution (e.g., a cell culture), followed by incubation or development, and then an observable result is detected using appropriate methods (e.g., spectroscopy). An indicator dye or stain can be used to facilitate detection of the observable result, and the solvent for dissolving the film can be e.g., a cell growth medium.

FIG. 6 is a schematic of a pre-formed testing environment, in accordance with an example: (A) containing e.g., a cell culture can have a compound vapor (e.g., a drug vapor) introduced by jet printing (B), and then incubated or developed to produce an observable result (C).

FIG. 7(A) illustrates formation of a compound vapor in an inert carrier gas by heating, in accordance with an example; and FIG. 7(B) is a top view of an array of Rhodamine 6G solutions produced by a jetting method, in accordance with an example.

FIG. 8(A) illustrates one embodiment of a vapor jet apparatus in accordance with an example; and FIG. 8(B) is a top view of an array of Rhodamine 6G solutions produced by a jetting method, in accordance with an example.

FIG. 9 is a plot of a concentration curve of Rhodamine 6G solutions formed by jet printing Rhodamine 6G vapor into solution from low to high jet dwell times (forward) or high to low jet dwell times (backward), as well as 10 μM deposited into a well before addition of solution (“direct jet”).

FIG. 10 is a schematic showing a process of vapor jet printing of a film on posts which are then introduced into a receiving vessel containing a solution.

FIG. 11 is a plot of a concentration curve of Rhodamine 6G solutions formed by jet printing Rhodamine 6G vapor into solution from low to high jet dwell times (forward) or high to low jet dwell times (backward), as well as printing of a film on posts prior to introduction into a receiving vessel containing a solution (“pre-jet”).

FIG. 12 illustrates one embodiment of a vapor jet printer capable of depositing a number of different, small-molecule APIs and excipients onto a variety of substrates. The insets show a multi-nozzle assembly permitting the side-by side or stacked film printing, as well as rapid cartridge loading with tamper-proof, hermetically sealed insert containing the compound.

FIG. 13 illustrates an organic vapor jet deposition (OVJP) system for depositing small molecular drugs.

FIG. 14 is a schematic of an high-throughput screening system deploying the vapor deposition device of FIG. 1, in accordance with an example.

FIG. 15 is a flow diagram illustrating the process flow of a high-throughput process that may be implemented by the high-throughput screening system of FIG. 14, in accordance with an example.

FIG. 16 illustrates a nozzle used in a vapor jet deposition system for use in vapor jetting of drug and imaging molecules as may be implemented by the high-throughput screening system of FIG. 14, in accordance with an example.

FIG. 17 illustrates a microscope/spectroscopy module as may be used in the high-throughput screening system of FIG. 14 and implementing an observation process of the FIG. 15, in accordance with an example. In the microscope/spectroscopy module, 1 is a fiber optic cable with varying diameter (50-100 μm) for pinhole limitation, 2 is a fiber zoom housing (for focusing spot size, 18 mm travel), 3 is an XY stage for fiber sample spot adjustment (microscale precision), 4 is a 90/10 (R:T) visible beamsplitter for image acquisition, 5 is a CMOS camera: monochromatic or color, 6 is a variable R:T UV/Vis/NIR/MIR beamsplitter depending on the imaging application needed (rotating splitter mount possible as well), 7 is a variable light source for fluorescent/visible/infrared imaging; a laser source or interferometer is needed for Raman/FTIR, respectively, 8 is an objective zoom housing (for focusing objective on a sample, 50 mm travel for varying sample thicknesses sizes, 4 mm travel housing possible for fine focusing), and 9 is a variable microscope objective with refractive (10-50×) and reflective (20×) options depending on the imaging system needed.

FIG. 18 shows images of substrates formed which may be desirable to use with the high-throughput system, in accordance with an example. FIGS. 18(A)-(D) are images of an SEM analysis of a fibrillar mat and porous 3D scaffolds with or without decellularized ECM. FIGS. 18(A) and (B) illustrate an electrospun fiber mat with (B) or without (A) decellularized ECM. Porogen-leached 3D scaffolds with (D) and without ECM (C), are shown in FIGS. 18(D) and (C), respectively. FIG. 18(E) illustrates several contemplated micro or nanostructures for the substrate, in accordance with an example.

FIG. 19 shows additional substrates which may be desirable to use with the high-throughput system, in accordance with an example. Sheets of plastic are patterned with an electrode material and a biocompatible material, then scored and folded to form 96 pillars, dimensioned to fit into a typical 96-well assay plate. This substrate can be further coated with a compound for investigation, such as a drug, in accordance with an example. FIG. 19A shows top and bottom views of the substrate, and FIG. 19B shows a top view displaying the pillars described above.

### DETAILED DESCRIPTION

Described herein are methods and apparatuses for manufacturing systems for high-throughput applications.

In certain aspects, described herein is a method of depositing a compound on a substrate comprising: (a) forming a compound vapor; (b) passing the compound vapor through a nozzle oriented towards the substrate; (c) depositing the compound on the substrate; (d) displacing one of the nozzle or the substrate relative to the other; and (e) repeating steps (c) and (d) as required to achieve a desired deposition protocol.

In certain aspects, described herein is a testing environment having a printed surface, wherein the printed surface is prepared by: (a) forming a compound vapor; (b) passing the compound vapor through a nozzle oriented towards the surface; (c) depositing the compound on the substrate as a solid; (d) displacing one of the nozzle or the substrate relative to the other; and (e) repeating steps (c) and (d) as required to achieve a desired deposition protocol.

In certain aspects, described herein is a method of screening an analyte comprising contacting the analyte with a testing environment described herein. In some cases, the testing environment is contacted with a solvent prior to contacting the analyte.

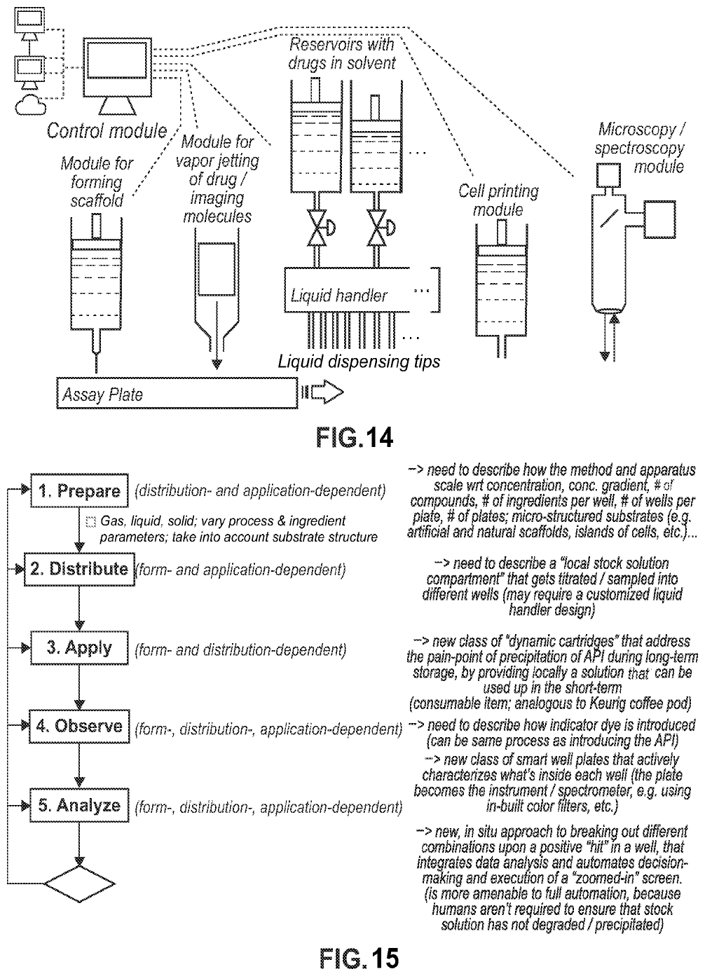

In certain aspects, described herein is a testing station comprising: a compound reservoir configured to hold a compound vapor; a nozzle array coupled to the compound reservoir, the nozzle array comprising a plurality of nozzles configured to receive the compound vapor; a substrate positioned to receive the compound vapor from the nozzle array, the substrate having one or more deposition regions configured for deposition of the compound on the substrate; and a positioner for displacing at least one of the nozzle array or the substrate relative to the other for depositing the compound on the substrate according to a designed deposition protocol. For example, a testing station as disclosed herein can be a high-throughput screening system as shown in FIG. 14.

In some cases, forming the compound vapor comprises subliming the compound optionally in the presence of a carrier gas. In some cases, forming the compound vapor comprises subliming the compound in the presence of the carrier gas within the nozzle. In some cases, forming the compound vapor comprises vaporizing a solution of the compound in a solvent. In some cases, forming the compound vapor comprises vaporizing the solution of the compound in the solvent within the nozzle.

In some cases, the compound vapor is formed by subliming a compound optionally in the presence of a carrier gas. In some cases, the carrier gas is an inert gas. In some cases, the carrier gas is selected from nitrogen, carbon dioxide, krypton, argon, hydrogen, helium, oxygen, water, methane, nitrous oxide or a mixture thereof. In some cases, the carrier gas is selected from nitrogen, argon, and helium.

In some cases, the compound is in the form of a dry powder prior to sublimation. In some cases, the compound is a drug. In some cases, the compound is deposited on the substrate as a solid. In some cases, the substrate comprises a liquid. In some cases, the substrate comprises a solid surface. In some cases, the substrate comprises a readable indicator identifying the one or more depositions regions and/or the desired deposition protocol. In some cases, the compound forms a thin film on the surface. In some cases, the thin film is substantially free of water. In some cases, the film is suitable for long-term storage with little or no degradation of the compound.

In some cases, the solvent comprises an organic solvent. In some cases, the solvent comprises water.

In some cases, the surface comprises a well, a post, a microwell plate, a Petri dish, an artificial scaffold, or a natural scaffold. In some cases, the surface comprises a stent, a balloon, or a catheter.

In some cases, the testing environment is suitable for long-term storage with little or no degradation of the compound.

In some cases, the deposition regions are a plurality of confined regions formed in the substrate.

In some cases, the testing station further comprises an adaptor interface configured to communicate the compound vapor from the compound reservoir to the nozzle array, the nozzle array being removably mounted to the adaptor interface. In some cases, the adaptor interface is configured to receive a plurality of different nozzle arrays, each different nozzle array being configured with nozzles have distinct nozzle heads from each other different nozzle array for performing different depositions of the compound on the substrate. In some cases, the testing station further comprises a controller configured to determine, from the readable indicator, which of the plurality of different compound vapors to provide to the nozzle array. In some cases, the testing station further comprises a characterization attachment. In some cases, the characterization attachment is a microscope or a micro spectroscopy module.

In some cases, the compound reservoir comprises a plurality of different compound vapors.

In some cases, the nozzle array comprises a readable indicator identifying a nozzle type for the nozzle array.

### Methods of Film Formation

Provided herein are methods of (a) forming a compound vapor; (b) passing the compound vapor through a nozzle oriented towards the substrate; (c) depositing the compound on the substrate as a solid; (d) displacing one of the nozzle or the substrate relative to the other; and (e) repeating steps (c) and (d) as required to achieve a desired deposition protocol.

The methods provided herein can comprise an inorganic compound or an organic compound. In some cases, the methods provided herein comprise an organic compound. The methods provided herein can comprise an organic compound that is between 100 and 5000 g/mol. In embodiments, the organic compound can be between approximately 100 and 1000 g/mol, for example, about 100 g/mol, about 150 g/mol, about 200 g/mol, about 250 g/mol, about 300 g/mol, about 400 g/mol, about 500 g/mol, about 600 g/mol, about 700 g/mol, about 800 g/mol, or about 900 g/mol. In some cases, the organic compound of the disclosure herein can have a vapor pressure of 10⁻³ Pascals to 10⁶ Pascals, such as, 10⁻² Pascals to 10⁵ Pascals, or 1 Pascals to 10⁴ Pascals.

In some cases, the organic compound can comprise a drug, e.g., an active pharmaceutical ingredient (“API”). In embodiments, the API can comprise a compound selected from the group consisting of caffeine, acetaminophen (paracetamol), carbamazepine, 5-methoxy sulfadiazine, ethenzamide, nalidixic acid, isoniazid, furosemide, sulfadimidine, celecoxib, temozolamide, piroxicam, tryptamine, chlorzoxazone, p-coumaric, itraconazole, fluoxetine, telaprevir, sildenafil, theophylline, aceclofenac, 5-nitrouracil, indomethacin, aripiprazole, and atorvastatin, or a mixture thereof.

In some cases, the organic compound is not an API. In various embodiments, the organic compound can comprise an agricultural and/or food industry-relevant compound. In some cases, the compound can comprise a nutritional or food compound, a nutraceutical compound, a cosmetic or personal care compound, a fragrance compound, a colorant or dye, an ink, a paint, and the like, by way of non-limiting example. In some cases, the organic compound can comprise a pesticide, antibacterial, confectionary, seasoning, glaze or a mixture thereof. In some embodiments, the organic compound can comprise gibberellin, benzylideneacetone, or acesulfame.

The present disclosure thus provides a solid film, for example, a deposited organic compound, such as a pharmaceutical active agent or a new chemical entity, patterned on a surface of a substrate. In certain variations, the surface has a continuous surface coating or film of the organic compound, while in other variations, the organic compound may be applied to select discrete regions of the surface. High quality films or coatings of low molecular organic compounds are formed by the processes according to certain aspects of the present disclosure that have high purity levels. For example, in certain variations, a purity level in one or more regions where of the compound is deposited may be greater than or equal to about 90% by mass of the compound, optionally greater than or equal to about 95% by mass, optionally greater than or equal to about 97% by mass, optionally greater than or equal to about 98% by mass, and in preferred aspects, optionally greater than or equal to about 99% by mass, optionally greater than or equal to about 99.5% by mass, optionally greater than or equal to about 99.7% by mass, and in certain variations, greater than or equal to about 99.99% by mass purity concentration. In certain variations, multiple compounds are present that together or cumulatively have the same purity levels. The deposited solid film may have a surface feature morphology ranging from molecularly flat to high surface area (e.g., a nanostructured surface) with feature sizes in the micrometer or nanometer regimes.

In certain aspects, methods of achieving solid films with high levels of purity and solubility are provided. For example, in certain variations, a solvent-free vapor deposition method is provided that includes depositing an organic compound on one or more discrete regions of a substrate in a process that is substantially free of solvents. By “substantially free” it is meant that solvent compounds or species are absent to the extent that undesirable and/or detrimental effects are negligible or nonexistent. In certain aspects, a vapor deposition process that is substantially free of solvents has less than or equal to about 0.5% by weight, optionally less than or equal to about 0.1% by weight, and in certain preferred aspects, 0% by weight of the undesired solvent species present during the deposition process.

A deposited organic compound may then be present at high purity levels, for example, at greater than or equal to about 99 mass % as described above, in the one or more discrete regions. The process for depositing the organic compound may be selected from the group consisting of: vacuum thermal evaporation (VTE), organic vapor jet printing (OVJP), organic vapor phase deposition (OVPD), organic molecular beam deposition (OMBD), molecular jet printing (MoJet), organic vapor jet printing (OVJP), and organic vapor phase deposition (OVPD).

In certain aspects, such a method may include entraining the organic compound in an inert gas stream or vacuum that is substantially free of any solvents prior to the depositing. An inert gas stream can comprise one or more generally nonreactive compounds, such as nitrogen, argon, helium, and the like. In certain variations, the inert gas stream comprises nitrogen.

Because many organic compounds, such as small molecular medicines, have sufficiently high vapor pressures (e.g., from about 1 Pa to about 100 Pa) and relatively low evaporation enthalpies (e.g., 100-300 kJ/mole), high evaporation rates (on the order of grams/(sec*m²)) can be achieved at temperatures of 100°-500° C., without reaching the temperature range where degradation of the compound can occur, even when evaporating at atmospheric pressure. Any process/system that enables deposition of molecular material onto a substrate from a vapor phase, where a source of the molecular material is a solid that evaporates or sublimates, can be used for forming the deposited organic compound pharmaceutical substances. This includes, but is not limited to: vacuum thermal evaporation (VTE), organic vapor phase deposition (OVPD), organic molecular beam deposition (OMBD), and molecular jet printing (MoJet).

However, the processes are not limited to solid sources of the compound. In certain aspects, prior to the entraining, the organic compound is in a form selected from the group consisting of: a powder, a pressed pellet, a porous material, and a liquid, e.g., a solution of a compound. In certain aspects, prior to the entraining, the organic compound is dispersed in pores of a porous material. In other aspects, prior to the entraining, the organic compound is dispersed in a liquid bubbler through which the inert gas stream passes. In yet other aspects, the entraining of the organic compound in the inert gas stream or vacuum is conducted by heating a source of a solid organic compound to sublimate or evaporate the organic compound.

A parameter of the deposition process may be adjusted to control or affect a morphology, a degree of crystallinity, or both the morphology and the degree of crystallinity of the deposited solid organic compound. The parameter is selected from the group consisting of: system pressure, a flow rate of the inert gas stream, a composition of the inert gas, a temperature of a source of the organic compound, a composition of the substrate, a surface texture of the substrate, a temperature of the substrate, and combinations thereof.

In certain aspects, a specific surface area of the deposited organic compound is greater than or equal to about 0.001 m²/g to less than or equal to about 1,000 m²/g. The deposited organic compound may be amorphous. When the deposited organic compound is amorphous, it may further define interconnected particles having an average particle size (e.g., average particle diameter) of greater than or equal to about 2 nm to less than or equal to about 200 nm. In other aspects, the deposited organic compound is crystalline or polycrystalline. In such variations, an average crystal size or domain may be greater than or equal to about 2 nm to less than or equal to about 200 nm.

In certain aspects, the one or more discrete regions on which the organic compound is deposited are continuous so that a solid film is formed on the surface of the substrate. In certain variations, the one or more discrete regions of the surface have a high surface area morphology, which may optionally define one or more nanostructures or microstructures. In certain variations, the films are flat (roughness <100 nm). In certain variations, the films comprise nanostructures. “Nano-sized” or “nanometer-sized” as used herein are generally understood by those of skill in the art to have at least one spatial dimension that is less than about 50 μm (i.e., 50,000 nm) and optionally less than about 10 μm (i.e., 10,000 nm). In certain aspects, the deposited organic compound defines a nanostructured surface having a plurality of nanostructures having a major dimension of greater than or equal to about 5 nm to less than or equal to about 10 μm. The resulting morphology depends on thermophysical properties of the organic compound, the substrate material and deposition conditions. The plurality of nanostructures may have a shape selected from the group consisting of: needles, tubes or cylinders, rods, platelets, round particles (although they need not be perfectly round or circular), droplets, fronds, tree-like or fern-like structures, fractals, hemispheres, puddles, interconnected puddles, islands, interconnected islands, and combinations thereof. The shape of nanostructures formed depends on the organic compound being deposited or the substrate, as well as the deposition process conditions, and film thickness.

In certain variations, a purity level of the deposited organic compound in the one or more discrete regions is any of those described previously, for example, greater than or equal to about 99.5 mass %. Suitable organic compounds, which may be pharmaceutical active ingredients or new chemical entities, may include by way of non-limiting example, various drugs or potential drugs (e.g., new chemical entities), including anti-proliferative agents; anti-rejection drugs; anti-thrombotic agents; anti-coagulants; antioxidants; free radical scavengers; nucleic acids; saccharides; sugars; nutrients; hormones; cytotoxin; hormonal agonists; hormonal antagonists; inhibitors of hormone biosynthesis and processing; antigestagens; antiandrogens; anti-inflammatory agents; non-steroidal anti-inflammatory agents (NSAIDs); antimicrobial agents; antiviral agents; antifungal agents; antibiotics; chemotherapy agents; antineoplastic/anti-miotic agents; anesthetic, analgesic or pain-killing agents; antipyretic agents, prostaglandin inhibitors; platelet inhibitors; DNA de-methylating agents; cholesterol-lowering agents; vasodilating agents; endogenous vasoactive interference agents; angiogenic substances; cardiac failure active ingredients; targeting toxin agents; and combinations thereof. The description of these suitable organic compounds/pharmaceutical active ingredients/new chemical entities is merely exemplary and should not be considered as limiting as to the scope of compounds or active ingredients which can be applied to a surface according to the present disclosure, as all suitable organic molecules and/or active ingredients known to those of skill in the art for these various types of compositions are contemplated. Furthermore, an organic compound may have various functionalities and thus, can be listed in an exemplary class above; however, may be categorized in several different classes of active ingredients.

Various suitable active ingredients are disclosed in Merck Index, An Encyclopedia of Chemicals, Drugs, and Biologicals, Thirteenth Edition (2001) by Merck Research Laboratories and the International Cosmetic Ingredient Dictionary and Handbook, Tenth Ed., 2004 by Cosmetic Toiletry and Fragrance Association, and at http://www.drugbank.ca/, the relevant portions of each of which are incorporated herein by reference. Each additional reference cited or described herein is hereby expressly incorporated by reference in its respective entirety. In certain variations, the organic compound is an active ingredient compound selected from the group: caffeine, (E)-3-(4-Methylphenylsulfonyl)-2-propenenitrile, fluorescein, paracetamol, ibuprofen, tamoxifen, cannabidiol, and combinations thereof. BAY 11-7082 ((E)-3-(4-Methylphenylsulfonyl)-2-propenenitrile) selectively and irreversibly inhibits transcription factor NF-κB activation (which otherwise regulates expression of inflammatory cytokines, chemokines, immunoreceptors, and cell adhesion molecules) and can inhibit TNF-α-induced surface expression of adhesion molecules ICAM-1, VCAM-1, and E-selectin in human endothelial cells.

In certain variations, the deposited organic compound has an enhanced rate of dissolution in comparison to a comparative powder or pellet form of the same deposited organic compound. Thus, a dissolution rate of the deposited organic compound in an aqueous solution (e.g., approximating physiological conditions) is at least ten times greater than a comparative dissolution rate of the comparative powder or pellet form of the deposited organic compound. In certain variations, a dissolution rate of the deposited organic compound in an aqueous solution is at least fifteen times greater, optionally twenty times greater, and optionally thirty times greater than a comparative dissolution rate of the powder or pellet form of the deposited organic compound.

In certain aspects, a solid film having a high surface area morphology can be formed by a modified organic vapor jet printing (OVJP) process, which eliminates the need for organic solvents and improves dissolution rates for small molecular-based organic materials, like APIs. The organic compound(s) that may be deposited by the OVJP process have relatively s and thus are considered to be organic compounds. OVJP processes utilize a carrier gas (e.g., nitrogen) to transport sublimated organic vapor towards a cooled substrate or other target in the form of a focused gas jet. The OVJP process enables scalable patterning of relatively small molecular materials.

In certain aspects, the present disclosure thus contemplates a solid film comprising greater than or equal to about 99 mass % of a deposited organic active ingredient compound having a molecular weight of less than or equal to about 1,000 g/mol. For example, the deposited organic compound may have a molecular weight of greater than or equal to about 100 g/mol to less than or equal to about 900 g/mol. The organic active ingredient compound is preferably a pharmaceutical active or a new chemical entity. The organic active ingredient is any of the compounds described above. By way of example, the deposited organic active ingredient compound may be selected from the group consisting of: anti-proliferative agents; anti-rejection drugs; anti-thrombotic agents; anti-coagulants; antioxidants; free radical scavengers; nucleic acids; saccharides; sugars; nutrients; hormones; cytotoxin; hormonal agonists; hormonal antagonists; inhibitors of hormone biosynthesis and processing; antigestagens; antiandrogens; anti-inflammatory agents; non-steroidal anti-inflammatory agents (NSAIDs); antimicrobial agents; antiviral agents; antifungal agents; antibiotics; chemotherapy agents; antineoplastic/anti-miotic agents; anesthetic, analgesic or pain-killing agents; antipyretic agents, prostaglandin inhibitors; platelet inhibitors; DNA de-methylating agents; cholesterol-lowering agents; vasodilating agents; endogenous vasoactive interference agents; angiogenic substances; cardiac failure active ingredients; targeting toxin agents; and combinations thereof. In certain variations, the deposited organic active ingredient compound is selected from the group consisting of: caffeine, (E)-3-(4-Methylphenylsulfonyl)-2-propenenitrile, fluorescein, paracetamol, ibuprofen, tamoxifen, and combinations thereof.

In certain aspects, the solid film has a specific surface area of the solid film that is greater than or equal to about 0.001 m2/g to less than or equal to about 1,000 m2/g. In certain variations, the deposited organic active ingredient compound in the solid film is amorphous. The solid film may further define particles having an average particle size of greater than or equal to about 2 nm to less than or equal to about 200 nm. Where the solid film is amorphous, the deposited organic active ingredient compound in the solid film is stable for greater than or equal to about 1 month, optionally greater than or equal to about 2 months, optionally greater than or equal to about 3 months, optionally greater than or equal to about 6 months, optionally greater than or equal to about 9 months, and in certain variations, optionally greater than or equal to about 1 year.

In other variations, the deposited organic active ingredient compound in the solid film is crystalline or polycrystalline. An average crystal size may be greater than or equal to about 2 nm to less than or equal to about 200 nm. An average thickness of the solid film may be less than or equal to about 300 nm and an average surface roughness (Ra) of the solid film is less than or equal to about 100 nm.

In other variations, an average thickness of the solid film is greater than or equal to about 300 nm. An average surface roughness (Ra) is greater than or equal to about 100 nm. The film having such a thickness defines a nanostructured surface comprising a plurality of nanostructures having a major dimension of greater than or equal to about 5 nm to less than or equal to about 10 μm. In such an embodiment, the plurality of nanostructures may have a shape selected from the group consisting of: needles, tubes, rods, platelets, round particles, droplets, fronds, tree-like structures, fractals, hemispheres, puddles, interconnected puddles, islands, interconnected islands, and combinations thereof.

Source temperature can be determined via thermogravimetry and tuned to obtain local deposition rate of approximately 0.5 μg/min. The temperature range and carrier gas rate can change depending on system size and configuration.

In certain embodiments, the solid film may comprise a deposited organic compound comprising caffeine. The plurality of nanostructures can have a particular structure, e.g., a needle shape or a tube shape. An average diameter of the plurality of nanostructures can be greater than or equal to about 5 nm to less than or equal to about 10 μm and an average length of greater than or equal to about 5 nm to less than or equal to about 100 μm.

In certain other embodiments, the solid film may comprise a deposited organic compound comprising (E)-3-(4-Methylphenylsulfonyl)-2-propenenitrile (BAY 11-7082). The plurality of nanostructures has a platelet shape, where an average height of the plurality of nanostructures is greater than or equal to about 10 nm to less than or equal to about 10 μm. An average width of the plurality of nanostructures is greater than or equal to about 5 nm to less than or equal to about 10 μm. An average length of greater than or equal to about 5 nm to less than or equal to about 100 μm.

In yet other embodiments, the solid film may comprise a deposited organic compound comprising fluorescein.

In yet further embodiments, the solid film may comprise a deposited organic compound comprising paracetamol. The plurality of nanostructures has a shape selected from the group consisting of: droplet, hemisphere, puddle, interconnected puddle, island, interconnected island, and combinations thereof, wherein an average major dimension of the plurality of nanostructures is greater than or equal to about 5 nm to less than or equal to about 20 μm.

In other aspects, the deposited organic compound according to the present teachings has an enhanced rate of dissolution as compared to a comparative powder or pellet form of the organic active ingredient. A dissolution rate of the deposited organic active ingredient compound in the solid film in an aqueous solution is at least ten times greater than a comparative dissolution rate of the comparative powder or pellet form of the organic active ingredient. The dissolution rate improvement may be any of those previously discussed above.

The films disclosed herein also have improved stability as compared to a comparative film of the active ingredient. In some cases, the films are substantially free of water or other solvents, reducing the rate and extent of degradation of the film.

In certain aspects, the solid film is substantially free of any binders or impurities. A solid film that is substantially free of binders or impurities has less than or equal to about 0.5% by weight, optionally less than or equal to about 0.1% by weight, and in certain preferred aspects, 0% by weight of the undesired binders or impurities present in the solid film composition. In certain variations, the solid film comprises greater than or equal to about 99.5 mass % of the deposited organic active ingredient compound; however, any of the purity levels discussed above may likewise be achieved in the solid film.

In certain aspects, the deposited organic compound on the surface is crystalline or polycrystalline. In other aspects, the deposited organic compound is amorphous. In this manner, substantially pure molecular medicinal films are fabricated that may have high surface area morphologies. The deposited organic compound exhibits enhanced solubility and stability.

In other variations, the present disclosure contemplates a solid film comprising multiple deposited organic active ingredient compounds each having a molecular weight of less than or equal to about 1,000 g/mol. The organic active ingredient compounds are preferably a pharmaceutical active or a new chemical entity. The organic active ingredient compounds are any of the compounds described above. A collective amount of the multiple organic active ingredient compounds may be greater than or equal to about 99 mass % in the solid film. The solid films may have any of the compositions or features described just above, which will not be repeated herein for brevity.

The methods provided herein can comprise subliming the compound to form a compound vapor. In some cases, the organic compound is sublimed at a temperature that is 1° C. to 300° C. above its onset of sublimation, as determined by thermogravimetric analysis (“TGA”), for example, 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 25° C., 30° C., 35° C., 40° C., 45° C., 50° C., 60° C., 70° C., 80° C., 90° C., 100° C., 150° C., 200° C., 250° C., or 300° C. above its onset of sublimation. In some cases, the organic compound is sublimed at a temperature that is 5° C. to 150° C., 10° C. to 100° C., or 10° C. to 50° C. above its onset of sublimation, as determined by TGA. In some embodiments, the compound vapor is supersaturated. In various cases, the compound vapor is in the presence of a carrier gas.

A carrier gas can comprise an inert gas (e.g., nitrogen, argon, CO₂, etc.) or a reactive gas (e.g. HCl, H₂O, O₃, CH₄, O₂ etc.). In some cases, the carrier gas can comprise nitrogen, carbon dioxide, krypton, argon, hydrogen, helium, oxygen, water, methane, nitrous oxide or a mixture thereof. In some cases, the carrier gas has a flow rate of about 1 to 500 standard cubic centimeters per minute (“sccm”). In embodiments, the carrier gas can have a flow rate of about 1 to 200 sccm, such as, about 1 to 150 sccm, about 1 to 100 sccm, about 1 to 50 sccm, or about 25 to 50 sccm, for example, 1 sccm, 5 sccm, 10 sccm, 15 sccm, 20 sccm, 25 sccm, 30 sccm, 35 sccm, 40 sccm, 45 sccm, 50 sccm, 60 sccm, 70 sccm, 80 sccm, or 90 sccm. In various embodiments, the carrier gas can carry the organic compound to a mixing chamber.

In some cases, the method provided herein can further comprise exposing the carrier gas to a guard force gas, such that the guard force gas surrounds the carrier gas, and the carrier gas comprises the vapor mixture. In some embodiments, the guard force gas comprises nitrogen, carbon dioxide, kypton, argon, hydrogen, helium, methane, nitrous oxide, or a mixture thereof.

The methods provided herein comprise condensing the vapor mixture onto a substrate to form the film. In embodiments, the method herein can further comprise moving the vapor mixture over the substrate. In some embodiments, the nozzle can be rastered over the substrate such that the vapor mixture moving over the substrate. In some embodiments, the nozzle can be rastered keeping the center to center line spacing from 0.01 mm to 100 mm, such as, 0.01 mm, 0.05 mm, 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, or 10 mm. In various embodiments, the nozzle can be rastered such that the raster velocity is from 0.01 mm/s to 100 mm/s. In some embodiments, the raster velocity is from 0.01 mm/s to 10 mm/s, for example, 0.01 mm/s, 0.05 mm/s, 0.1 mm/s, 0.2 mm/s, 0.3 mm/s, 0.4 mm/s, 0.5 mm/s, 0.6 mm/s, 0.7 mm/s, 0.8 mm/s, 0.9 mm/s, 1 mm/s, 2 mm/s, 3 mm/s, 4 mm/s, 5 mm/s, 6 mm/s, 7 mm/s, 8 mm/s, 9 mm/s, or 10 mm/s. In some cases, the method can further comprise moving the substrate under the vapor mixture.

In some embodiments, the substrate can be at a temperature of −100° C. to 100° C., for example, −90° C., −80° C., −70° C., −60° C., −50° C., −40° C., −30° C., −20° C., −10° C., −5° C., 0° C., 5° C., 10° C., 15° C., 20° C., 25° C., 30° C., 35° C., 40° C., 45° C., 50° C., 55° C., 60° C., 65° C., 75° C., 80° C., 85° C., 90° C., or 95° C. In some cases, the substrate can be at a temperature of −50° C. to 50° C., such as, 5° C. to 25° C. The substrate can be made of any material that one of skill in the art would find suitable to coat with the film.

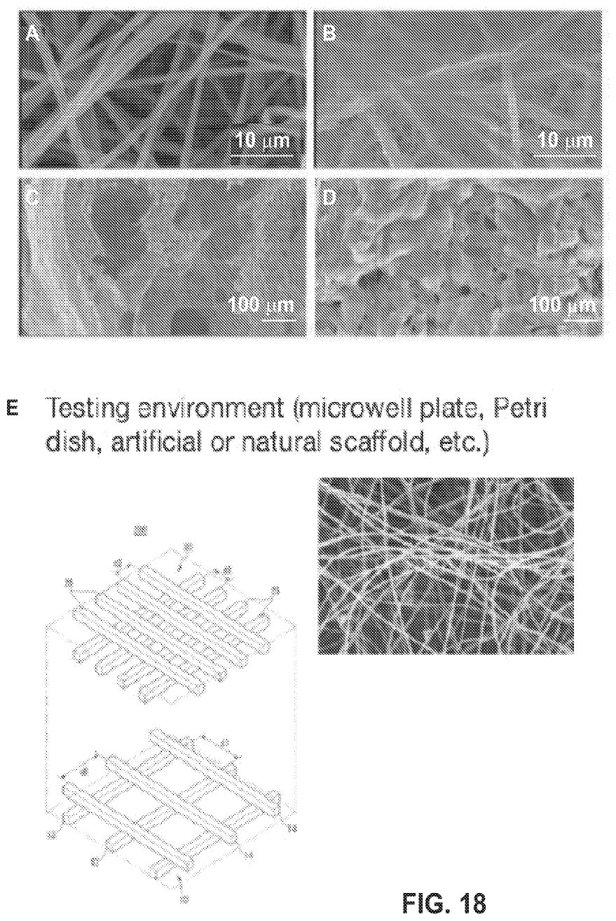

As used herein, the term “substrate” refers to substances including but not limited to metal, plastic, wood, ceramic, quartz, glass, paper, composites, semiconductors, or the like, or a mixture thereof, or an aqueous or non-aqueous solvent, e.g., an organic solvent. In embodiments, the substrate can comprise quartz, glass, metal, plastic, ceramic or a combination thereof. In some cases, the substrate can be a well plate, such as a microwell plate, a well, a post, a Petri dish, an artificial scaffold, or a natural scaffold. In various cases, the substrate can comprise a medical device. In various cases, the medical device is selected from the group consisting of a stent, balloon, needle, microneedle, syringe, cannula, catheter, sponge, clip, mesh, bandage, gauze, dressing, tape, swab, burn dressing, staple, implant, contact lens, medical tubing, adhesive patches, and endoscopic device. In various cases, the medical device is a stent, shunt, balloon, needle, catheter, or a sponge. In some cases, the substrate can be water, an aqueous solution (e.g., a buffer), or an organic solvent (e.g., methanol, ethanol, isopropanol, dichloromethane, chloroform, tetrahydrofuran, tetrahydropyran, dimethylsulfoxide, dimethylformamide, hexanes, benzene, toluene, xylene, diethyl ether, and the like). Substrates described herein can have a defined micro- or nano-structure, as shown in FIG. 18.

### Printed Testing Environments

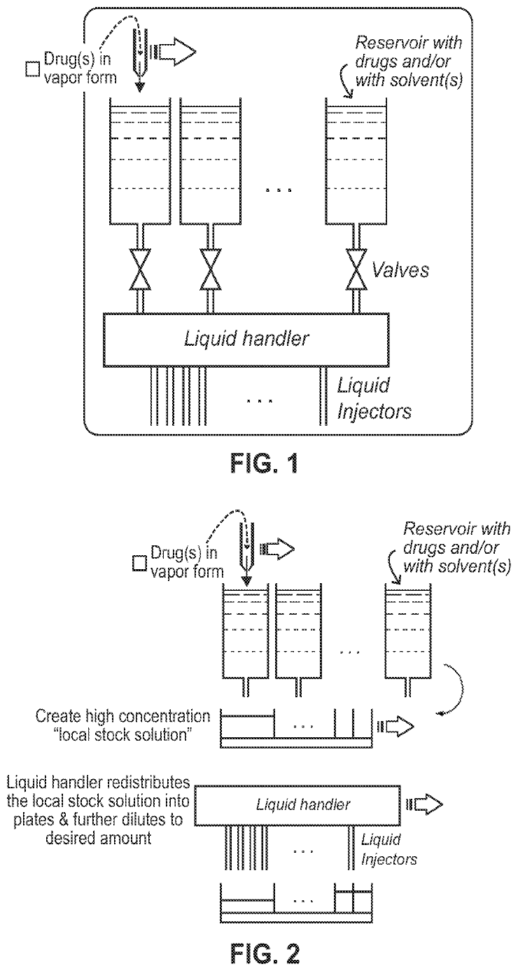

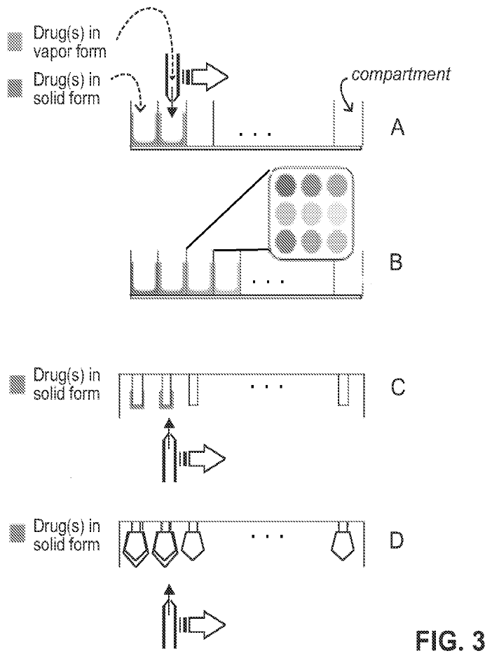

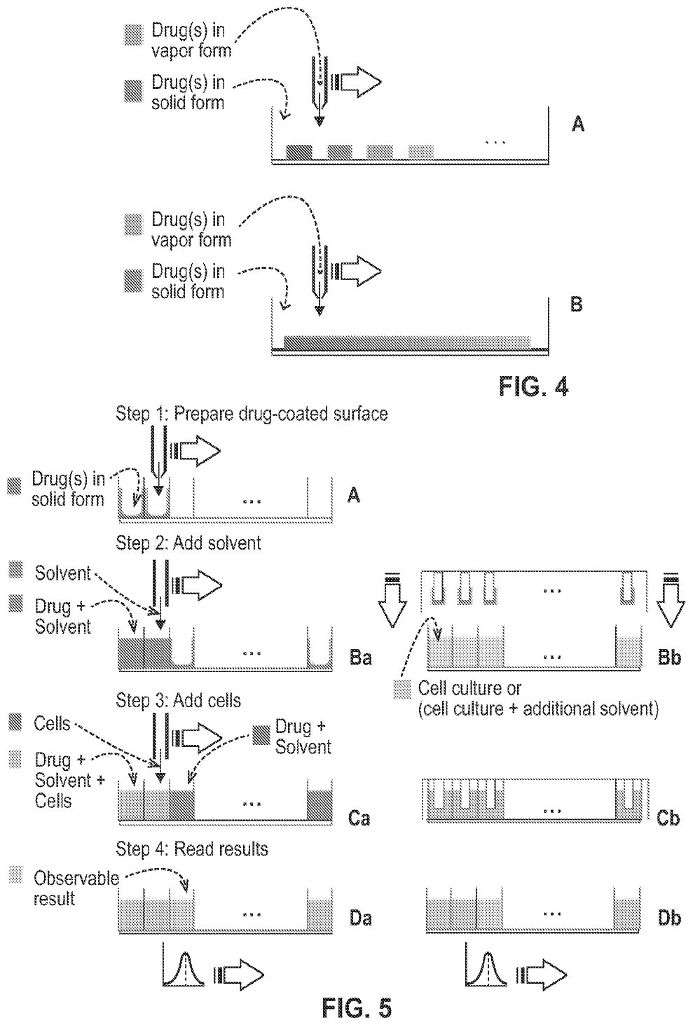

Also described herein are printed articles comprising a solid deposited film comprising a compound, e.g., an organic compound or a drug, or a pharmaceutical composition comprising at least one organic compound. The solid deposited films may have any of the composition or features described above. The printed articles can be prepared as in FIGS. 1-4.

In some cases, an article is provided that includes a surface of a solid substrate having one or more discrete regions patterned with a deposited organic compound having a molecular weight of less than or equal to about 1,000 g/mol. The organic compound is any of the compounds described above. The deposited organic compound is present at greater than or equal to about 99 mass % in the one or more discrete regions. In certain aspects, the one or more discrete regions of the surface are continuous and the deposited solid organic compound forms a solid film on the surface of the pharmaceutically acceptable substrate.

In other cases, an article is provided that includes a surface of a solid substrate having one or more discrete regions patterned with multiple deposited organic compounds each having a molecular weight of less than or equal to about 1,000 g/mol. The organic compounds are any of the compounds described above. The multiple deposited organic compounds are cumulatively present at greater than or equal to about 99 mass % in the one or more discrete regions. Thus, any of the solid films described above may be disposed on a surface of a solid substrate. Further, the solid substrate may be as described just above.

In yet other cases, the present disclosure provides an article comprising a pharmaceutically acceptable substrate defining a surface. The materials selected for the substrate are preferably pharmaceutically acceptable or biocompatible, in other words, substantially non-toxic to cells and tissue of living organisms. Pharmaceutically acceptable materials may be those which are suitable for use in contact with the tissues of humans and other animals without resulting in excessive toxicity, irritation, allergic response, or other problems or complications, commensurate with a reasonable benefit/risk ratio. The article also includes a deposited solid pharmaceutical active ingredient having a molecular weight of less than or equal to about 1,000 g/mol. A pharmaceutical active ingredient is a drug or other compound operable for the prevention or treatment of a condition or disorder in a human or other animal, the prevention or treatment of a physiological disorder or condition, or to provide a benefit that outweighs potential detrimental impact in a conventional risk-benefit assessment. The organic active ingredient may be any of those described above. Thus, the articles and compositions of the present disclosure may be used for the treatment or prevention of systemic disorders, such as cancer, autoimmune diseases, cardiovascular disease, stroke, diabetes, severe respiratory infection, inflammation, pain control, and the like.

The deposited solid pharmaceutical active ingredient is present at greater than or equal to about 99 mass % in one or more discrete regions on the surface of the pharmaceutically acceptable substrate. The one or more discrete regions of the surface are continuous and the deposited solid pharmaceutical active ingredient forms a solid film on the surface of the pharmaceutically acceptable substrate. Thus, any of the solid films described above having a pharmaceutical active ingredient may be disposed on a surface of a solid substrate.

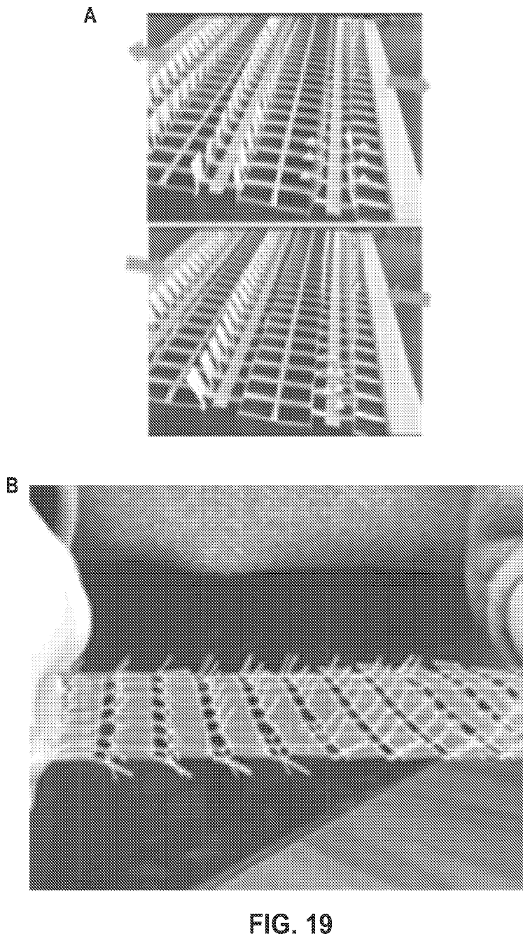

In certain aspects, the pharmaceutically acceptable substrate is biodegradable. By biodegradable, it is meant that the materials forming the substrate dissolve or erode upon exposure to a solvent comprising a high concentration of water, such as serum, growth or culture media, blood, bodily fluids, or saliva. In some variations, a substrate may disintegrate into small pieces or may disintegrate to collectively form a colloid or gel. In certain variations, the pharmaceutically acceptable substrate comprises a pharmaceutically acceptable material selected from the group consisting of: glass, metals, siloxanes, polymers, hydrogels, organogels, organic materials, natural fibers, synthetic fibers, ceramic, biological tissue, and combinations thereof. In other variations, the pharmaceutically acceptable material is selected from the group consisting of: glass, metals, siloxanes, polymers, hydrogels, organogels, natural fibers, synthetic fibers, and combinations thereof. The deposited solid pharmaceutical active ingredient can be formed on any type of substrate geometry, including flat substrates, microneedles, spheres, tubes, curved surfaces, meshes, and the like. Further, the substrate can be of any size. In certain non-limiting variations, the pharmaceutically acceptable substrate is selected from the group consisting of: a microneedle, medical equipment, an implant, a film, such as a dissolvable film or a film having a removable backing, a gel, a patch, a dressing like a gauze, a non-adhesive mesh, a bandage, a membrane, a foil, a foam, or a tissue adhesive, a fabric, such as a woven, nonwoven, or knitted fabric, a sponge, a stent, a contact lens, a subretinal implant prosthesis, dentures, braces, a wearable device, a bracelet, and combinations thereof. In some aspects, the substrate is a flexible material, e.g., a plastic, which can be cut and folded to form surfaces suitable for depositing a solid film thereupon. An example of flexible substrates which can be cut and folded to form surfaces suitable for depositing a solid film thereupon is shown in FIG. 19.

In other aspects, the present disclosure contemplates an article comprising a solid deposited film comprising a pharmaceutical composition. The pharmaceutical composition comprises at least one organic compound having a molecular weight of less than or equal to about 1,000 g/mol. In certain variations, the pharmaceutical composition further comprises at least one additional deposited compound distinct from the organic compound, so that a plurality of organic compounds are co-deposited to form a solid deposited film. Thus, the pharmaceutical composition may comprise at least two organic compounds. In certain variations, the pharmaceutical composition has at least one organic compound present at greater than or equal to about 99 mass % in the solid deposited film.

The printed article described herein can be a testing environment having a printed surface. In some cases, the printed surface is prepared by: (a) forming a compound vapor; (b) passing the compound vapor through a nozzle oriented towards the surface; (c) depositing the compound on the substrate as a solid; (d) displacing one of the nozzle or the substrate relative to the other; and (e) repeating steps (c) and (d) as required to achieve a desired deposition protocol. Illustrative examples of printed substrates formed using this method and their properties are shown in FIGS. 7-11.

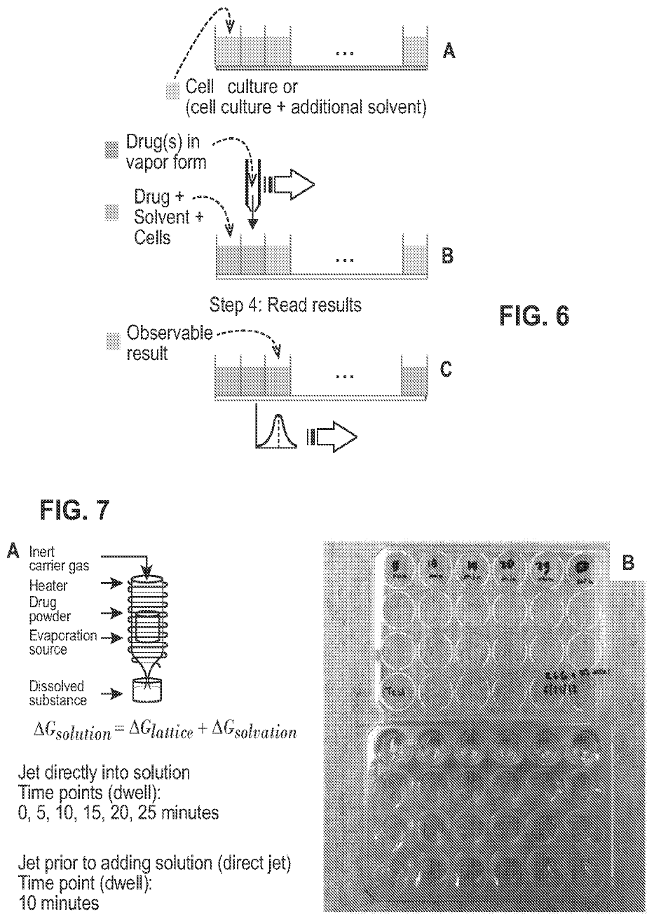

The testing environments described herein are suitable for screening methods of various throughput levels. Illustrative examples of tests or assays that use the screening methods disclosed herein are shown in FIGS. 5 and 6. For example, the testing environments can be used in low-throughput methods, medium-throughput methods, and high-throughput methods. For example, the testing environments are useful for testing 1 to 10, 1 to 100, 1 to 1,000, 1 to 10,000, 1 to 100,000, or 1 to 1,000,000 compounds per test or assay. In some cases, the testing environments are useful for testing 1 to 10, 10 to 20, 20 to 30, 30 to 40, 40 to 50, 50 to 60, 60 to 70, 70 to 80, 80 to 90, or 90 to 100 compounds per test or assay. In some cases, the testing environments are useful for testing 1 to 100, 1 to 200, 1 to 300, 1 to 400, 1 to 500, 1 to 600, 1 to 700, 1 to 800, 1 to 900, or 1 to 1,000 compounds per test or assay. In some cases, the testing environments are useful for testing 1 to 1,000, 1 to 2,000, 1 to 3,000, 1 to 4,000, 1 to 5,000, 1 to 6,000, 1 to 7,000, 1 to 8,000, 1 to 9,000, or 1 to 10,000 compounds per test or assay. In some cases, the testing environments are useful for testing 1 to 10,000, 1 to 20,000, 1 to 30,000, 1 to 40,000, 1 to 50,000, 1 to 60,000, 1 to 70,000, 1 to 80,000, 1 to 90,000, or 1 to 100,000 compounds per test or assay. In some cases, the testing environments are useful for testing 1 to 100,000, 1 to 200,000, 1 to 300,000, 1 to 400,000, 1 to 500,000, 1 to 600,000, 1 to 700,000, 1 to 800,000, 1 to 900,000, or 1 to 1,000,000 compounds per test or assay.

The testing environments described herein are suitable for high-throughput screening methods. Such high-throughput screening methods are suitable for identifying and quantifying specific molecules in a sample. In addition, the assays can be used for drug screening in a high throughput screening of compounds having suitable binding affinity to an analyte such as a biomolecule, e.g., a protein of interest (see, e.g., Geysen et al., 1984, PCT application WO84/03564). In some testing environments described herein, large numbers of different small test compounds are synthesized as thin films on a solid substrate. The test compounds are reacted with e.g., identified genes, or fragments thereof, and washed. Bound molecules are then detected by detection methods known in the art.

The testing environments described herein can be used in screening assays to identify, from a library of diverse molecules, one or more compounds having a desired activity, e.g., modulating the amount of a target molecule. A “screening assay” is a selective assay designed to identify, isolate, and/or determine the structure of, compounds within a collection that have a preselected activity. By “identifying” it is meant that a compound having a desirable activity is isolated, its chemical structure is determined (including without limitation determining the nucleotide and amino acid sequences of nucleic acids and polypeptides, respectively) the structure of and, additionally or alternatively, purifying compounds having the screened activity). Biochemical and biological assays are designed to test for activity in a broad range of systems ranging from protein-protein interactions, enzyme catalysis, small molecule-protein binding, to cellular functions. Such assays include automated, semi-automated assays and HTS (high throughput screening) assays.

In HTS methods such as those described herein, many discrete compounds can be tested in parallel by robotic, automatic or semi-automatic methods so that large numbers of test compounds are screened for a desired activity simultaneously or nearly simultaneously. It is possible to assay and screen up to about 6,000 to 20,000, and even up to about 100,000 to 1,000,000 different compounds a day using the integrated systems described herein.

In some cases, screening comprises contacting each cell culture with a diverse library of member compounds, some of which are ligands of the target, under conditions where complexes between the target and ligands can form, and identifying which members of the libraries are present in such complexes. In another non limiting modality, screening comprises contacting a target enzyme with a diverse library of member compounds, some of which are inhibitors (or activators) of the target, under conditions where a product or a reactant of the reaction catalyzed by the enzyme produce a detectable signal. In the latter modality, inhibitors of target enzyme decrease the signal from a detectable product or increase a signal from a detectable reactant (or vice-versa for activators).

The testing environments disclosed herein can be used for screening a plurality of test compounds. In certain embodiments, the plurality of test compounds comprises between 1 and 200,000 test compounds, between 1 and 100,000 test compounds, between 1 and 1,000 test compounds, between 1 and 100 test compounds, or between 1 and 10 test compounds. In certain embodiments, the test compounds are provided by compound libraries, whether commercially available or not, using combinatorial chemistry techniques. In certain embodiments, the compound libraries are immobilized in the wells of a well plate. In some embodiments, the compound libraries are immobilized on posts which may be inserted into the wells of a well plate.

High throughput screening can be used to measure the effects of drugs on complex molecular events such as signal transduction pathways, as well as cell functions including, but not limited to, cell function, apoptosis, cell division, cell adhesion, locomotion, exocytosis, and cell-cell communication. Multicolor fluorescence permits multiple targets and cell processes to be assayed in a single screen. Cross-correlation of cellular responses will yield a wealth of information required for target validation and lead optimization.

In another aspect, the present disclosure provides a method for analyzing the contents of wells or cells comprising providing a testing environment which contain multiple wells or cells, wherein the wells or cells contain one or more fluorescent reporter molecules; scanning multiple wells or cells in each of the locations containing wells or cells to obtain fluorescent signals from the fluorescent reporter molecule in the wells or cells; converting the fluorescent signals into digital data; and utilizing the digital data to determine the distribution, environment or activity of the fluorescent reporter molecule within the wells or cells.

The testing environments described herein can comprise a microarray. Microarrays may be prepared, used, and analyzed using methods known in the art (see, e.g., Brennan et al., 1995, U.S. Pat. No. 5,474,796; Schena et al., 1996, Proc. Natl. Acad. Sci. U.S.A. 93: 10614-10619; Baldeschweiler et al., 1995, PCT application WO95/251116; Shalon, et al., 1995, PCT application WO95/35505; Heller et al., 1997, Proc. Natl. Acad. Sci. U.S.A. 94: 2150-2155; and Heller et al., 1997, U.S. Pat. No. 5,605,662). In some cases, a microarray comprises drugs, peptides, or other desired molecules which can be assayed to identify a candidate agent.

### Printed Testing Stations

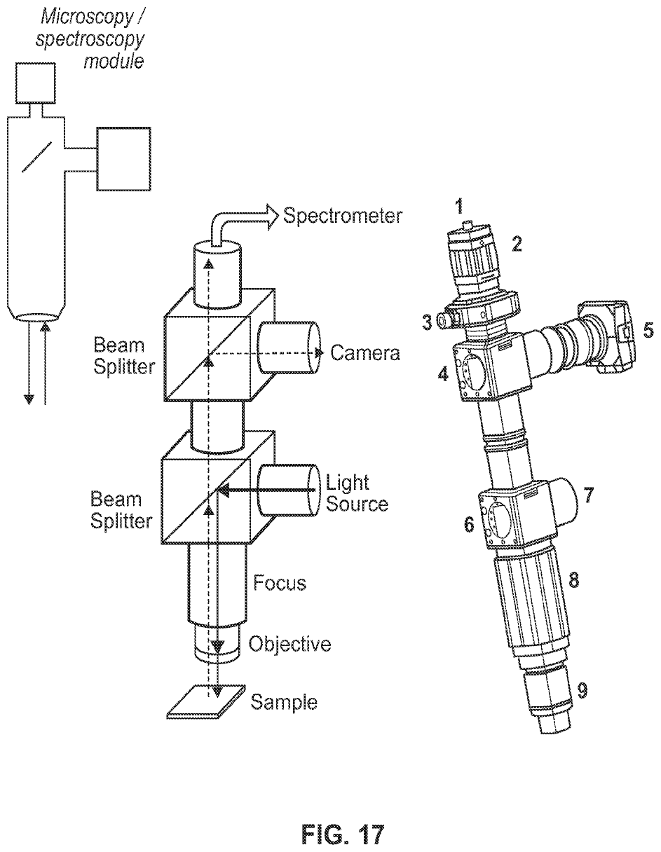

The methods described herein can also be used in a testing station a compound reservoir configured to hold a compound vapor; a nozzle array coupled to the compound reservoir, the nozzle array comprising a plurality of nozzles configured to receive the compound vapor; a substrate positioned to receive the compound vapor from the nozzle array, the substrate having one or more deposition regions configured for deposition of the compound on the substrate; and a positioner for displacing at least one of the nozzle array or the substrate relative to the other for depositing the compound on the substrate according to a designed deposition protocol. Illustrative examples of testing stations as described herein and their methods of use are shown in FIGS. 12-17.

The testing stations described herein comprise a testing environment as described herein, e.g., a microarray or well plate which has been printed with a film as described herein. In some cases, the testing station includes a detection device, such as a microscope, a spectrometer, or a camera. In some cases, the testing station comprises a UV/Vis spectrometer, an infrared spectrometer, a Raman spectrometer, a mass spectrometer, or the like.

In some cases, the compound reservoirs comprises a plurality of different compound vapors. In some cases, the compound vapors comprise different carrier gases. In some cases, the compound vapors comprise the same carrier gas.

In some cases, the substrate comprises a readable indicator identifying the one or more deposition regions and/or the desired deposition protocol. In some cases, the readable indicator comprises a bar code or a quick response (QR) code.

### Apparatus for Printing Films

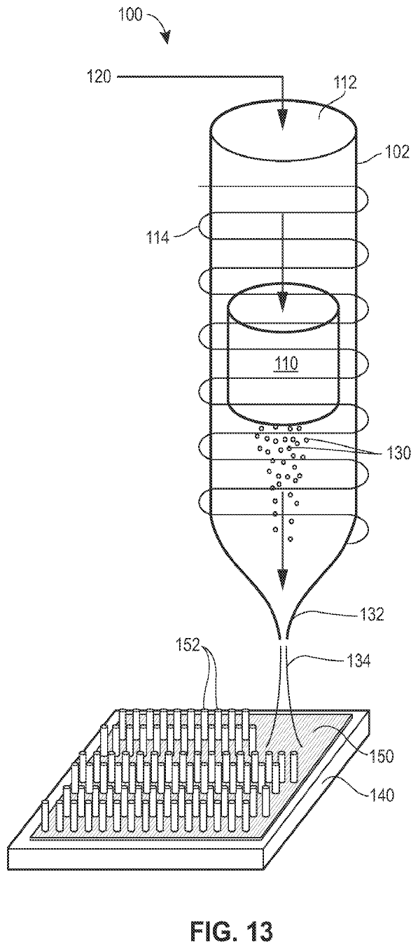

In certain cases, a printed film of the instant disclosure is fabricated with an organic vapor jet printing (OVJP) system 100 like that shown in FIG. 13. A cylindrical reactor 102 contains a source 110 of the organic compound. The source 110 is in a solid form of the organic compound (e.g., a powder or a pressed pellet). The source 110 may hold or contain the organic compound, for example, as a porous material having the organic compound distributed within pores. The reactor 102 has an inlet 112 in which an inert carrier gas stream 120 enters. A heater 114 is disposed about the exterior or may be otherwise integrated into the reactor 102. A material in the evaporation source 110 is sublimed or evaporated and carried by the inert carrier gas 120. The method thus comprises entraining an organic compound in an inert carrier gas stream 120 by heating the source 110 to sublimate or evaporate the compound 130, so that it is a vapor form and entrained in the inert carrier gas stream 120. The entraining can occur by passing the inert carrier gas stream 120 over, by, or through the source 120. Controllable system parameters include carrier gas rate (sccm), evaporation source temperature (° C.), and substrate temperature (° C.). As shown in FIG. 13, the compound 130 in the inert carrier gas stream 120 is directed through a nozzle 132 in a focused jetted stream 134 towards a substrate 140. The nozzle 132 is translated above the substrate via xyz motion controllers, enabling printing of any desired deposit pattern.

The substrate 140 may be a solid or a liquid. The substrate 140 may be formed of a material like glass, metals, siloxanes, polymers, hydrogels, organogels, natural fibers, synthetic fibers, and any combinations thereof. As will be described further below, the substrate 140 may be a microneedle, medical equipment, an implant, a film, a gel, a patch, a dressing, a fabric, a bandage, a sponge, a stent, a contact lens, a subretinal implant prosthesis, dentures, braces, a wearable device, a bracelet, a well plate, a microarray, and combinations thereof. When the substrate 140 is a liquid, it may be a polar or non-polar liquid, including aqueous liquids. The liquid may comprise one or more solvents.

In an example, methods further include condensing the compound 130 as it contacts the substrate 140 on one or more discrete regions. In this manner, the surface of the substrate 140 may be selectively patterned by directing the jetted stream 134 towards desired regions (or the surface may be temporarily masked). In the variation shown in FIG. 13, the one or more discrete regions of the surface of the substrate 140 are continuous and the compound forms a solid film 150 on the surface of the substrate 140. In certain aspects, the film deposited by OVJP onto the one or more discrete regions of the substrate 140 may have a loading density of greater than or equal to about 1×10⁻⁴ g/cm² to less than or equal to about 1 g/cm². In certain variations, a specific surface area of the film on the substrate 140 surface is greater than or equal to about 0.001 m²/g to less than or equal to about 1000 m²/g.

Thicknesses may vary depending on the amount of time that the jetted stream 134 is directed at a particular area of the substrate 140 surface where the compound accumulates. In certain variations, when an average thickness of the solid film 150 of compound in the one or more discrete regions is less than or equal to about 300 nm, an average surface roughness (Ra) of the surface profile (the two-dimensional profile of the surface taken perpendicular to the lay, if any) is less than or equal to about 100 nm. As noted above, for solid films 150 with a thickness of 200±100 nm, the films are generally flat with a surface roughness of less than about 100 nm. Starting with a thickness of around 200±100 nm, undulations occur in a solid film 150, which further produces and define a plurality of nanostructures 152. In this manner, the surface of the solid film 150 is nanostructured.

While the solid film 150 may have any of the purity levels previously described above, in certain variations, the condensed organic compound is present at greater than or equal to about 99.5 mass %.

Two variations of an OVJP apparatus and process are described herein. In one variation, deposition of the organic compound is performed at atmospheric pressure conditions, rather than pulling a moderate vacuum (10⁻³ Torr). Such a process can be conducted in a glove box with appropriate ventilation. In case of oxygen or moisture-sensitive organic compounds, the process can be performed in a glove box with inert gas environment. In other variations, the entraining and directing are conducted at reduced pressure conditions, for example, at greater than or equal to about 0.1 Torr to less than or equal to about 500 Torr.

In other aspects, a parameter of the OVJP process may be adjusted to affect a morphology, a degree of crystallinity, or both the morphology and the degree of crystallinity of the compound. The parameter may be selected from the group consisting of: system pressure, flow rate of the inert gas stream, inert gas composition, a temperature of the source, a composition of a target substrate, a surface texture of the target substrate, a temperature of the target substrate, and combinations thereof. The morphology may include the nanostructures formed. The compound in the solid film 150 may be amorphous. In other aspects, the compound in the solid film 150 is crystalline or polycrystalline. The compound may be any of those described previously above.

Thus, FIG. 13 shows an example schematic of an OVJP system/device for making films in accordance with certain variations of the present disclosure. The methods of fabricating a surface patterned with a compound, such as a pharmaceutical active agent, thus may include sublimating or otherwise volatilizing the compound contained in a source/target. Multiple devices may be used in parallel. A system may include one source or multiple sources holding heated small molecular medicine in a powder form. An inert carrier gas (e.g., nitrogen, argon or helium) is introduced to the device and directed towards the source/target of organic material. In certain variations, the compound is in solid form, for example, in the form of a powder. Heat is also applied within the system (for example, via a heater) so that the compound is sublimated or volatilized to a gas/fluid phase and carried by the inert carrier gas stream passing by. The carrier gas having entrained gaseous compound is then ejected from a nozzle in a form of focused jet and directed towards a substrate that has a controlled temperature (e.g., may be cooled), where the entrained small organic molecules are condensed. The material can be deposited with precise control of amount, with highly controlled weight ranges of 1×10⁻⁴ g/cm² to 0.1 g/cm², by way of example.

Such a method of fabrication is highly controllable. Various parameters may be controlled in such an OVJP system, including: pressure and flow rate, including carrier gas flow rate (sccm), inert carrier gas type, evaporation source temperature (° C.), and substrate composition, substrate surface texture, and substrate temperature (° C.), by way of example. Changes in each of the parameters can affect film morphology (e.g., features type, size, and distribution) and degree of crystallinity. The nozzle is translated above the substrate via xyz motion controllers, enabling printing of the organic material in any pattern, including a wide variety of preselected deposit patterns. The resolution of a pattern formed depends on nozzle geometry, inert gas type and flow conditions. In order to obtain a large area deposit, adjacent lines of the deposit can be printed with one nozzle or with multiple nozzles. This enables scalability of the process with robust process conditions. Further, in certain aspects, such a method desirably eliminates the requirement for liquid solvents, vacuum, or extensive post-processing steps to obtain a desired particle size for one or more organic compounds. Importantly, such an OVJP works without liquid solvents or vacuum, and allows for controlled degree of crystallization in the organic films.

In other aspects, the present disclosure contemplates a method for rapid dissolution of compounds. Compound vapor may be jetted directly into liquids. In certain aspects, the liquid may be an aqueous solution, demonstrating how precise drug concentrations can be rapidly reached, without the need for additional solvents and/or powder preparation. Solutions of small molecular organic compounds, for example, are used extensively in many industries: food, cosmetics/perfume, pharmaceuticals, printing and paints. As background, conventionally to achieve a given concentration of organic solute in original powder form, the required amount of powder is immersed directly in the solvent and is dissolved until all powder particles are separated into solvated molecules. This process is especially challenging for low solubility substances, where dissolution rate is very slow. To enhance dissolution rates, powder particle size is reduced (via milling or other methods), and solution is usually heated. This approach can be both time and energy consuming, as well as potentially damaging to the solvent.

An additional drawback of the conventional technique of direct immersion of powder solute in the solvent is when the actual needed concentration of a compound or solution volume is very low. For instance, if a desired concentration is on the order of micromolar, and volume needed is 10 ml, the weight needed for a 200 g/mole material would be on the order of micrograms. This weight is not feasible to measure accurately for a precursor powder, therefore a higher concentration of solution is made with subsequent dilution with additional amount of solvent. This process is undesirable from both economical and safety standpoint (when dealing with organic solvent).

### Other Embodiments

In some embodiments, described herein are methods for high-throughput screening and high-throughput testing, and systems for carrying out the methods. In some aspects, a high-throughput screening method described herein comprises a preparation stage, a distribution stage, an application stage, an observation stage, and an analysis stage.

In some embodiments, a testing station described herein can be a high-throughput screening system. In some cases, the high-throughput screening system can be a modular system, such as the system shown in FIG. 14. Such a system comprises multiple stages or modules each having a specific function, and which can be integrated to create a system useful for carrying out the methods described herein. For example, the high-throughput system of FIG. 14 comprises an assay plate which is passed in turn across a module for forming a scaffold, e.g., by 3D printing, a module for depositing a compound such as a drug or an imaging molecule like a dye, a liquid handling module further comprising liquid reservoirs holding a solvent and/or a solution of a compound such as a drug, and liquid dispensing tips, a module for printing or dispensing cells, and a module for analyzing the deposited elements, such as a microscope or a spectroscope or spectrometer. Each of the aforementioned modules of FIG. 14 are further linked to a control module, which allows for individual control and coordination of the modules. In some cases, the control module is a computer.

In some embodiments, high-throughput testing methods described herein can comprise one or more stages. In some cases, high-throughput testing methods can comprise stages as described in FIG. 15. In some cases, high-throughput testing methods comprise using an e-jet nozzle to make scaffold, using the nozzle to coat the scaffold with APIs or other compounds, using a cell printer attachment to seed cells onto the scaffold, using a pipette to deliver growth medium and liquid factors or fluorescent dyes, and using a micro-spectrometer to characterize the assay.

In some embodiments, methods comprise a preparation stage that involves e.g., gathering the requisite materials for printing a compound, preparing e.g., reagents or solutions for a printing method, and programming the control module to carry out a desired printing sequence and/or experiment. A preparation stage can comprise, for example, determining an appropriate apparatus scale, concentration of reagents and/or solutions, and the number of compounds for analysis. In a well-based analysis, a preparation stage can comprise determining the number of ingredients per well, number of wells per plate, number of plates, the micro- or nano-structure of the substrate, and the like.

In some examples, the control module stores in a computer-readable medium a high-throughput testing protocol having data fields identifying reagents and/or solutions and ingredients and compounds for analysis. The stored protocol may include data identifying the different modules/stages, such as those shown in FIG. 14, and parameters for controlling each of the different modules/stages. Such parameters may be those used in the processes identified in FIG. 15 as discussed further below, for example.

In some cases, the method comprises a distribution stage, such as introducing a prepared reagent or solution, e.g., a local stock solution, into a reservoir or compartment, e.g., a well. A distribution stage can comprise titrating or sampling a local stock solution into different wells, e.g., through a customized liquid handler. In some cases, a control module integrated into the system can determine or be instructed as to the nature of the HTS substrate or testing environment. In some cases, the HTS substrate or testing environment is an array or assay, such as those comprising a well plate. In some cases, the system can form the substrate or testing environment prior to or as part of the distribution stage. For example, the system can comprise a scaffold-forming module as shown in FIG. 14. In some cases, such a scaffold-forming module comprises a 3D printer. Suitable systems and processes for forming a scaffold can be those described in the art, such as those described in US Patent Publication US 2014/0296996 A1, the contents of which are incorporated herein in their entireties. A distribution stage can comprise analyzing design parameters, for example stored in a protocol, to determine the appropriate scaffold to form for a particular test or application, e.g., by optimizing the size, shape, material, number of layers, etc.

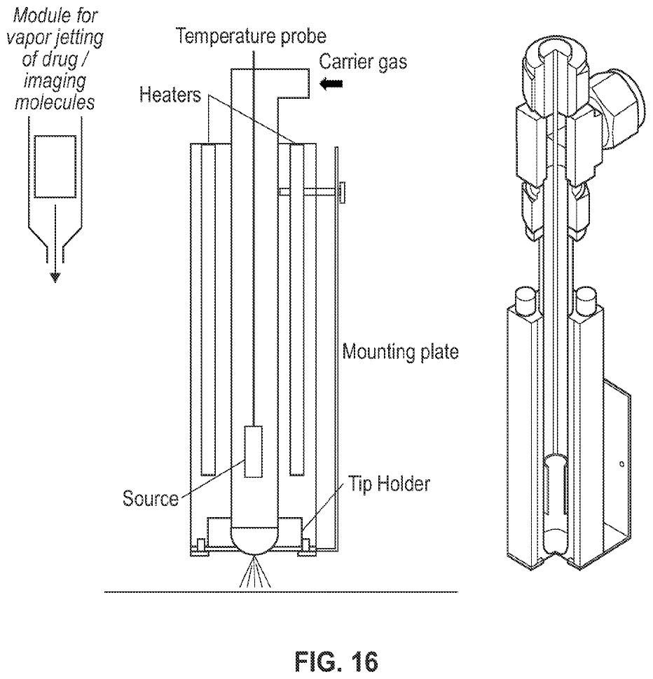

In some cases, the method comprises an application stage, e.g., jet printing or deposition, optionally followed by reorientation of a substrate relative to a printing means such as a nozzle, and repetition until a desired pattern is obtained. An application stage can comprise e.g., using a customized cartridge, such as a dynamic cartridge that provides a local solution intended for use in the near-term. The distribution and application stages can be selected based on the form and nature of the distribution of application. For example, the distribution and application stages can be appropriately modified if a compound is vapor jetted into a liquid or directly onto a surface to provide a film. An application stage can comprise a module for vapor jetting drug or imaging molecules, such as the module shown in FIG. 16. Such a module can comprise a source of a compound, heaters to vaporize the compound, a carrier gas inlet and flow channel, a temperature probe, and a mounting plate and tip holder to maintain the module in proper position for control of the vapor jetting application.

In some cases, the method comprises an observation stage, such as detection with a microscope or spectroscope. An observation stage can comprise observing a color change in an indicator dye which is present in a testing environment disclosed herein, and which dye can be introduced to the testing environment e.g., by vapor deposition or jetting. An observation stage can further comprise the use of a smart well plate which characterizes its contents via an integrated mechanism (e.g., through built-in color filters), without requiring a separate microscope or spectroscope.

In some cases, the method comprises an analysis stage, such as computer-assisted analysis of experimental data. An analysis stage can comprise configuring further combinations of test components based on computerized analysis of an initial test or screen. An analysis stage can comprise integrated data analysis that allows for automated decision-making and further analysis of a particular subset of initially-tested combinations of components or variables. An analysis stage can be partially automated, or fully automated.

In some examples, the analysis stage is implemented in part by the control module. For example, the control module may receive data from the analysis stage such as spectroscopic data from a microscopy/spectroscopy module providing response data from applying different drugs to different compounds. Based on the microscopy/spectroscopy response data, the control module may modify operation of any of the preceding stages in the high-throughput system of FIG. 14. For example, different scaffolding formations may be performed, based on the microscopy/spectroscopy response data to generate new substrates for testing. The control module may determine that different imaging molecules should be used based on the microscopy/spectroscopy response data. In some examples, the control module may be determine that different drug combinations should be generated by the vapor deposition device for testing of targets in the substrate/assay plate. The control module may be adjust different cell printing as well in response to the microscopy/spectroscopy response data.

The control module of FIG. 14 may be part of a high-throughput screening processing system and implemented on a computing device such as a computer, tablet or other mobile computing device, or server. The system may include a number of processors, controllers or other electronic components for processing data and performing the processes described herein. The system may be implemented on a computing device and in particular on one or more processing units, which may represent Central Processing Units (CPUs), and/or on one or more or Graphical Processing Units (GPUs), including clusters of CPUs and/or GPUs. Features and functions described for the system may be stored on and implemented from one or more non-transitory computer-readable media of the computing device. The computer-readable media may include, for example, an operating system and instructions for operating the high-throughput screening system with vapor deposition devices are described and illustrated herein. More generally, the computer-readable media may store protocols and other data, executable code, etc. use for implementing the techniques herein. The computing device may include a network interface communicatively coupled to a network, for communicating to and/or from a portable personal computer, smart phone, electronic document, tablet, and/or desktop personal computer, or other computing devices. The computing device further includes an I/O interface connected to devices, such as digital displays, user input devices, etc. In the illustrated example, the system is implemented on a single server. However, the functions of the system may be implemented across distributed devices connected to one another through a communication link. In other examples, functionality of the system may be distributed across any number of devices, including the portable personal computer, smart phone, electronic document, tablet, and desktop personal computer devices shown.

The network may be a public network such as the Internet, private network such as research institutions or corporations private network, or any combination thereof. Networks can include, local area network (LAN), wide area network (WAN), cellular, satellite, or other network infrastructure, whether wireless or wired. The network can utilize communications protocols, including packet-based and/or datagram-based protocols such as internet protocol (IP), transmission control protocol (TCP), user datagram protocol (UDP), or other types of protocols. Moreover, the network can include a number of devices that facilitate network communications and/or form a hardware basis for the networks, such as switches, routers, gateways, access points (such as a wireless access point as shown), firewalls, base stations, repeaters, backbone devices, etc.

The computer-readable media may include executable computer-readable code stored thereon for programming a computer (e.g., comprising a processor(s) and GPU(s)) to the techniques herein. Examples of such computer-readable storage media include a hard disk, a CD-ROM, digital versatile disks (DVDs), an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. More generally, the processing units of the computing device may represent a CPU-type processing unit, a GPU-type processing unit, a field-programmable gate array (FPGA), another class of digital signal processor (DSP), or other hardware logic components that can be driven by a CPU.

It is also contemplated that the apparatus and methods described herein are useful for analyzing substrates (e.g., compounds) which are difficult to analyze using traditional high-throughput screening methods. In some cases, the apparatus and methods are useful for screening compounds suitable for medium-throughput screening. In some cases, the apparatus and methods are useful for screening compounds suitable for low-throughput screening.

It is to be understood that while the disclosure is read in conjunction with the detailed description thereof, the foregoing description is intended to illustrate and not limit the scope of the disclosure, which is defined by the scope of the appended claims. Other aspects, advantages, and modifications are within the scope of the following claims.

### EXAMPLES

The following examples are provided for illustration and are not intended to limit the scope of the invention.

### Example 1: Liquid Jetting

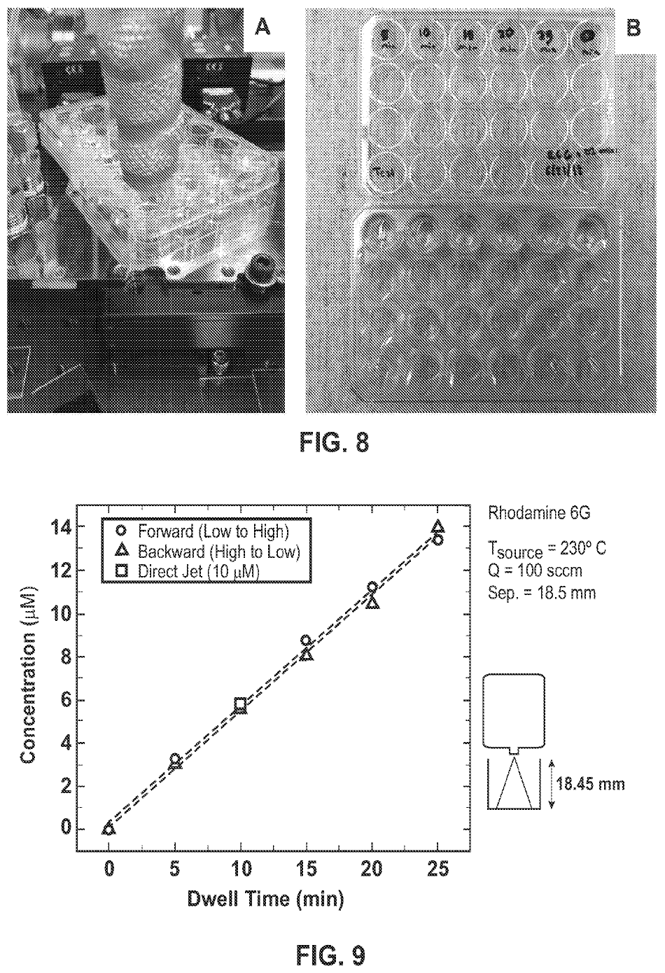

FIG. 7 shows the general process by which vapor jet deposition can be used to deliver organic small molecules directly into a liquid in a desired location on an assay plate, as opposed to onto a solid substrate or into a bulk liquid (e.g. in a beaker). Here, both some degree of localization is desired, yet it is undesirable to break the surface of the liquid, which can cause splashing. For the process conditions outlined here, the nozzle to substrate separation (in this case the surface of the liquid) is kept at 10 mm or greater, as shown in FIG. 8, whereas in performing organic vapor jet deposition onto a solid substrate this distance can be considerably shorter.

For absorbance measurement, an Ocean Optics dip probe was utilized, which requires a certain volume of liquid to be present in each well to completely cover the detector. These parameters enable the deposition in, e.g. a typical 24 well assay plate. As shown in FIG. 7, the concentration of solution was progressively increased by changing how long the nozzle dwell time above the surface of the liquid.

To create assay plates containing dry-form small molecular ingredients, the molecules were deposited first into the wells, without any solution present, as shown in FIG. 9. In this case, the nozzle was positioned directly above the empty well and the organic material was deposited as a film onto the bottom of the well plate. Following this, the solvent (deionized water) was added and the plate was left to sit for one hour to allow the material to dissolve. This experiment was only carried out for the 10 minute dwell time point, however as seen in the plot in FIG. 9, the concentration attained in both the liquid jetting and traditional OVJP scenarios is largely the same.

### Example 2: Organic Vapor Jet Deposition on Posts

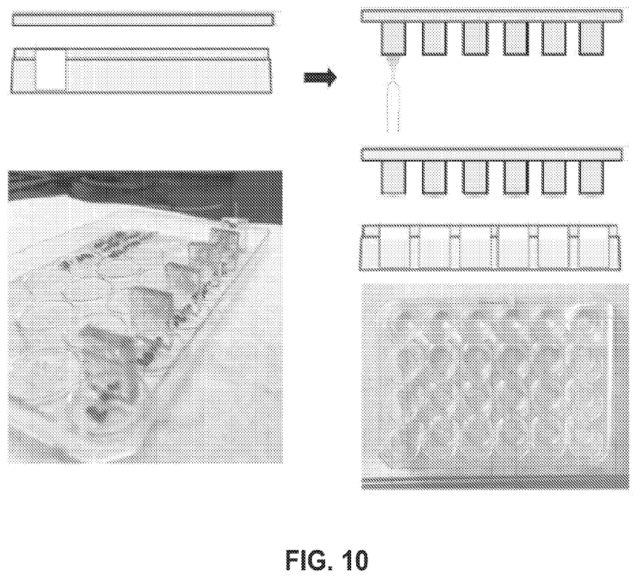

FIG. 10 illustrates how organic vapor jet deposition can be used to print organic small molecules onto “posts” prior to insertion into a well plate containing liquid solution. For the purposes of this experiment, the films were printed onto the posts prior to their attachment to the lid of the well plate. With sufficiently wide posts, a custom well plate lid is fabricated that already has structural features (e.g. posts) and print directly onto their surface. The posts in the illustrated example are acrylic, and were cleaned prior to deposition and handling. The concentration achieved upon dissolving the material in solution can be varied by adjusting the nozzle dwell time over each post.

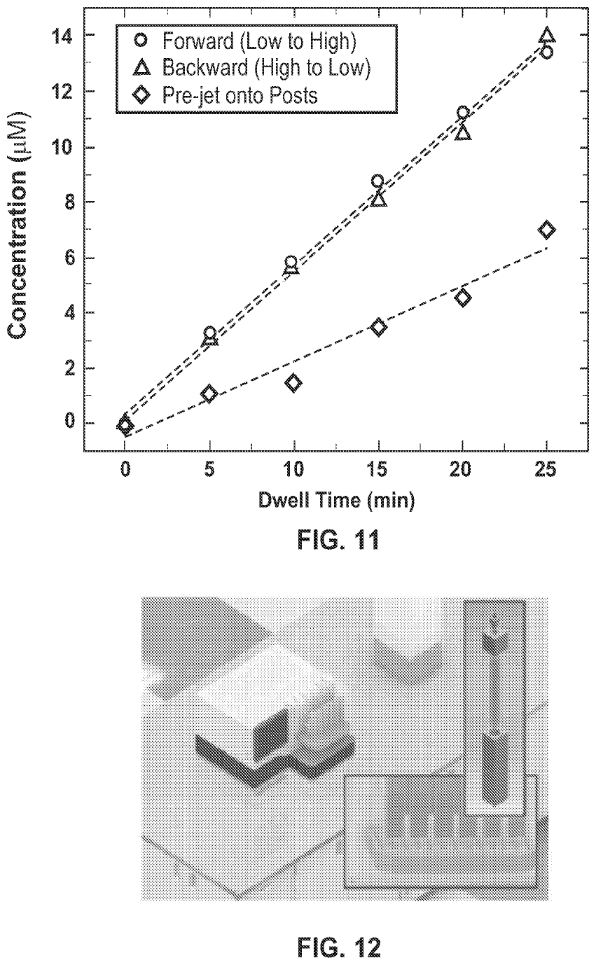

FIG. 11 shows a comparison between the examples of organic vapor jet deposition into liquid-filled wells described previously with jetting onto posts. Here, for each dwell time point, it can be seen that the amount of material dissolving off of the post is less than what was deposited via jetting into liquid, attributed to the post material having a glass transition temperature, T_{g}, below the source temperature of the nozzle and sufficient deformation of the surface of the post to trap some of the deposited material. This can be useful to further decrease the amount of material dissolved, as sometimes necessary. Different materials comprising the posts are selected, depending on the application; e.g. metal, glass, or a high temperature plastic to prevent degradation; or dissolvable material such as pollulene or a co-crystal to further enhance dissolution.

The foregoing description is given for clarity of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of several of the steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

We claim:

1. A method of depositing a compound on a substrate comprising:
(a) forming a compound vapor;
(b) passing the compound vapor through a nozzle oriented towards the substrate;
(c) depositing the compound on the substrate;
(d) displacing one of the nozzle or the substrate relative to the other; and
(e) repeating steps (c) and (d) as required to achieve a desired deposition protocol.

2. The method of claim 1, wherein forming the compound vapor comprises subliming the compound optionally in the presence of a carrier gas.

3. The method of claim 2, wherein the compound is in the form of a dry powder prior to sublimation.

4. The method of claim 2, wherein forming the compound vapor comprises subliming the compound in the presence of the carrier gas within the nozzle.

5. The method of claim 1, wherein forming the compound vapor comprises vaporizing a solution of the compound in a solvent.

6. The method of claim 4, wherein the solvent comprises an organic solvent.

7. The method of claim 4, wherein the solvent comprises water.

8. The method of claim 4, wherein forming the compound vapor comprises vaporizing the solution of the compound in the solvent within the nozzle.

9. The method of any one of claims 1 to 8, wherein the compound is deposited on the substrate as a solid.

10. The method of any one of claims 1 to 9, wherein the substrate comprises a liquid.

11. The method of any one of claims 1 to 10, wherein the substrate comprises a solid surface.

12. The method of any one of claims 1 to 11, wherein the compound is a drug.

13. A testing environment having a printed surface, wherein the printed surface is prepared by:
(a) forming a compound vapor;
(b) passing the compound vapor through a nozzle oriented towards the surface;
(c) depositing the compound on the substrate as a solid;
(d) displacing one of the nozzle or the substrate relative to the other; and
(e) repeating steps (c) and (d) as required to achieve a desired deposition protocol.

14. The testing environment of claim 13, wherein the surface comprises a well, a post, a microwell plate, a Petri dish, an artificial scaffold, or a natural scaffold.

15. The testing environment of claim 13 or 14, wherein forming the compound vapor comprises subliming the compound optionally in the presence of a carrier gas.

16. The testing environment of claim 15, wherein the compound is in the form of a dry powder prior to sublimation.

17. The testing environment of claim 13 or 14, wherein forming the compound vapor comprises vaporizing a solution of the compound in a solvent.

18. The testing environment of claim 17, wherein the solvent comprises an organic solvent.

19. The testing environment of claim 17, wherein the solvent comprises water.

20. The testing environment of any one of claims 13 to 19, wherein the compound is deposited on the substrate as a solid.

21. The testing environment of any one of claims 13 to 20, wherein the compound forms a thin film on the surface.

22. The testing environment of claim 21, wherein the compound is a drug.

23. The testing environment of claim 21 or 22, wherein the thin film is substantially free of water.

24. The testing environment of any one of claims 13 to 23, wherein the testing environment is suitable for long-term storage with little or no degradation of the compound.

25. The testing environment of any of claims 13 to 24, wherein the substrate is a stent, balloon, needle, microneedle, syringe, cannula, catheter, sponge, clip, mesh, bandage, gauze, dressing, tape, swab, burn dressing, staple, implant, contact lens, medical tubing, adhesive patch, or endoscopic device.

26. The testing environment of claim 25, wherein the substrate is a stent, balloon, or catheter.

27. A method of screening an analyte comprising contacting the analyte with a testing environment of any one of claims 13 to 26.

28. The method of claim 27, wherein the testing environment is contacted with a solvent prior to contacting the analyte.

29. The method of claim 27 or 28, wherein the method is a high throughput screening method.

30. The method of claim 29, comprising a high throughput screening of a plurality of test compounds.

31. The method of claim 30, wherein the plurality of test compounds are immobilized in the wells of a well plate.

32. The method of claim 30, wherein the plurality of test compounds are immobilized on posts.

33. A testing station comprising:
a compound reservoir configured to hold a compound vapor;
a nozzle array coupled to the compound reservoir, the nozzle array comprising a plurality of nozzles configured to receive the compound vapor;
a substrate positioned to receive the compound vapor from the nozzle array, the substrate having one or more deposition regions configured for deposition of the compound on the substrate; and
a positioner for displacing at least one of the nozzle array or the substrate relative to the other for depositing the compound on the substrate according to a designed deposition protocol.

34. The testing station of claim 33, wherein the substrate comprises a well, a post, a microwell plate, a Petri dish, an artificial scaffold, or a natural scaffold.

35. The testing station of claim 34, wherein the deposition regions are a plurality of confined regions formed in the substrate.

36. The testing station of claim 34, further comprising an adaptor interface configured to communicate the compound vapor from the compound reservoir to the nozzle array, the nozzle array being removably mounted to the adaptor interface.

37. The testing station of claim 34, wherein the adaptor interface is configured to receive a plurality of different nozzle arrays, each different nozzle array being configured with nozzles having distinct nozzle heads from each other different nozzle array for performing different depositions of the compound on the substrate.

38. The testing station of claim 37, wherein the compound reservoir comprises a plurality of different compound vapors.

39. The testing station of claim 38, wherein the nozzle array comprises a readable indicator identifying a nozzle type for the nozzle array.

40. The testing station of claim 39, wherein the substrate comprises a readable indicator identifying the one or more depositions regions and/or the desired deposition protocol.

41. The testing station of claim 39 or 40, further comprising a controller configured to determine, from the readable indicator, which of the plurality of different compound vapors to provide to the nozzle array.

42. The testing station of claim 33, wherein the deposition regions are a plurality of confined regions formed in the substrate.

43. The testing station of any one of claims 33 to 42, wherein the compound vapor is formed by subliming a compound optionally in the presence of a carrier gas.

44. The testing station of claim 43, wherein the compound is in the form of a dry powder prior to sublimation.

45. The testing station of claim 43 or 44, wherein forming the compound vapor comprises vaporizing a solution of the compound in a solvent.

46. The testing station of claim 45, wherein the solvent comprises an organic solvent.

47. The testing station of claim 45, wherein the solvent comprises water.

48. The testing station of any one of claims 33 to 47, wherein the compound is deposited on the substrate as a solid.

49. The testing station of any one of claims 44 to 48, wherein the compound forms a thin film on the surface.

50. The testing station of any one of claims 33 to 49, wherein the compound is a drug.

51. The testing station of claim 49 or 50, wherein the thin film is substantially free of water.

52. The testing station of any one of claims 49 to 51, wherein the film is suitable for long-term storage with little or no degradation of the compound.

53. The testing station of any one of claims 33 to 52, further comprising a characterization attachment.

54. The testing station of claim 53, wherein the characterization attachment is a microscope or a micro spectroscopy module.
